(19) United States
(12) Patent Application Publication
(10) Pub. No.: US 2021/0260362 A1
(43) Pub. Date: Aug. 26, 2021

(54) CARDIOVASCULAR DEVICE

(71) Applicant: Eucardia S.R.L., Milano (IT)

(72) Inventor: Roberto Parravicini, Modena (IT)

(21) Appl. No.: 17/253,861

(22) PCT Filed: Jun. 18, 2019
(86) PCT No.: PCT/IT2019/050144

§ 371 Date: Dec. 18, 2020

(30) Foreign Application Priority Data
Jun. 18, 2018 (IT) 102018000006417

(51) Int. Cl. A61M 60/837; A61M 60/268; A61M 60/17; A61M 60/451

(52) U.S. Cl. CPC A61M 60/837; A61M 60/268; A61M 2205/0266; A61M 60/451; A61M 2207/00; A61M 60/17

(57) ABSTRACT

The cardiovascular device (1) comprises: a flexible membrane (3) insertable in a transverse position in a ventricular cavity (10); a flexible frame (2) which supports the membrane (3) in the transverse position, the flexible frame (2) comprising a plurality of shaped thread-like elements (4) each of which comprises a first portion (4A) which has a first end that is centrally associated with other first ends (4A) of other first portions of other thread-like elements (4) forming a central hub (5) and a second portion (4B) which is helically shaped and which has having a second end opposite the first end which is attached to an attachment element (6) which is common to other second ends of other thread-like elements (4), which form all together a plurality of loops (7; 28) everted toward the outside to support and constrain the membrane (3).

### FIELD OF THE INVENTION

The invention concerns a cardiovascular device, generally usable to be implanted in a cardiac ventricle, typically the left ventricle, to correct cardio-dilatative pathologies thereof.

### BACKGROUND OF THE INVENTION

Cardiovascular devices are known that can be surgically implanted in one of the ventricles of the heart, precisely in the left ventricle, when, due to dilatative pathologies, the function of pumping blood toward the aorta becomes insufficient.

Typically, known cardiovascular devices comprise a flexible diaphragm which is surgically implantable inside the ventricle in a transverse position and which consists of a membrane supported by a reticular frame.

Both the membrane and also the frame that supports it are elastically flexible to spontaneously adapt to the systole and diastole movements of the heart and the outline of the membrane is maintained in hermetic contact with the internal walls of the ventricle to avoid unwanted leakages of blood between them and the membrane.

The frame that supports the membrane is typically made in the form of a kind of umbrella whose ribs open and flex during the systolic and diastolic phases and which extend radially from a central support rod which is also used for the surgical handling of the devices during the positioning steps.

Typically, the elasticity of these cardiovascular devices, after their final positioning, allows to follow the contractions and dilations of the walls of the ventricle without damage during the diastolic and systolic phases.

In this way, the overall volume of a ventricle can be reduced as needed in the presence of pathologies that cause an undesired dilation thereof, and the pumping function of the blood, normally performed by alternating movements of the ventricle, can be restored so as to reach the substantially normal volumetric characteristics.

A cardiovascular device is known from U.S. Pat. No. 8,684,906.

According to this patent, the cardiovascular device is suitable to be inserted transversely inside the left ventricle of the heart so as to reduce its overall volume in the presence of dilatative cardiopathies.

The device comprises a support structure and a membrane stretched on the support structure; both are made of elastically flexible materials in order to follow the systole and diastole movements of the heart.

The membrane and the support structure flex together between an active position of pushing the blood toward the aorta and an inactive return position, and the peripheral edge of the membrane is arranged substantially in sealed contact with the internal walls of the ventricle.

The support structure consists of a central rod from which some support arms protrude in a radial direction, each of which have a “petal-type” shape, all connected to each other in the central area and which, typically, support the membrane which is attached stretched above them.

According to this patent, it is provided that the central rod is divided into two coaxial sections, but detached from each other and that in correspondence with the facing ends of the two sections, two small magnets are mounted excitable in two conditions, precisely in a condition of reciprocal attraction and a condition of repulsion, this in order to facilitate the contraction and dilation movements of the support structure during the pulsating step of the membrane.

However, this state of the art has some disadvantages.

One disadvantage is that the particularly articulated structure of these cardiovascular devices, which have very small overall sizes, of the order of a few centimeters, is complex both from the point of view of production, and also from that of efficiency and maintenance of the functional characteristics over time.

Another disadvantage is that the materials that have to be used in the production are particularly expensive due to their specific technical characteristics, both in terms of biocompatibility, and also in terms of lightness, and also in terms of mechanical resistance, and therefore the total cost of a known cardiovascular device is high.

A further disadvantage is that the joints or the attachment points between the arms that support the membrane and the central rod that supports them can present functioning difficulties, or be subject to breakage due to the stress caused by the continuous cycles of contractions and dilations, compromising the functionality of the entire cardiovascular device and requiring its replacement to maintain the vital conditions of the patients.

### OBJECTS OF THE INVENTION

One object of the invention is to improve the state of the art.

Another object of the invention is to provide a cardiovascular device that has a simple and relatively economical structure.

A further object of the invention is to provide a cardiovascular device that comprises a limited number of joints and junctions, maintaining an effective functioning over time even when subjected to functional stress.

According to one aspect of the invention, a cardiovascular device is provided, in accordance with the characteristics of claim 1.

Other aspects of the invention are indicated in the dependent claims.

The invention allows to obtain the following advantages:
reduce the internal volume of a cardiac ventricle, in particular of the left ventricle, in the presence of dilatative pathologies that reduce its functional efficacy;
restore an effective pumping action of the blood toward the aorta and the entire circulatory system of patients;
spontaneously adapt to the systole and diastole movements of the ventricle;
be easily implantable and clampable, both surgically and also percutaneously, in a functioning position.

### BRIEF DESCRIPTION OF THE DRAWINGS

Further characteristics and advantages of the invention will become apparent from the following detailed description of preferred, but not exclusive, embodiments of a cardiovascular device, given as a non-restrictive example with reference to the attached drawings wherein:

FIG. 1 is a schematic lateral and transparent view of a cardiovascular device according to the invention, in a first embodiment;

FIG. 2 is a schematic and transparent view from above of the device of FIG. 1;

FIG. 3A is a detailed and interrupted view of a part of a membrane that forms the cardiovascular device of FIG. 1, in a greatly enlarged scale;

FIG. 3B is an interrupted schematic view in a greatly enlarged scale of a detail that shows the composition of the membrane of FIG. 3A;

FIG. 4 is a schematic view of the cardiovascular device of FIG. 1, implanted inside a ventricular cavity;

FIG. 5 is a schematic view of the cardiovascular device of FIG. 1 without the membrane and in a configuration ready to be implanted inside a ventricular cavity;

FIG. 6 is a schematic lateral and transparent view of a second possible embodiment of a cardiovascular device according to the invention;

FIG. 7 is a schematic and transparent view from above of the cardiovascular device of FIG. 6.

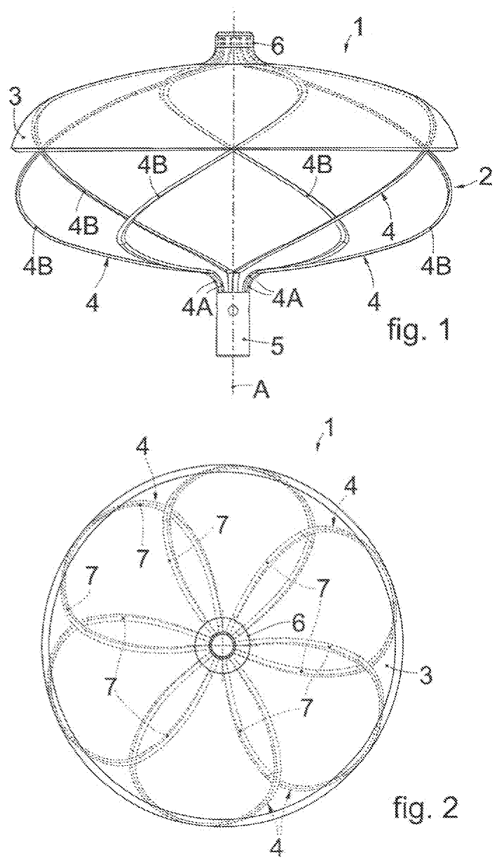

### DETAILED DESCRIPTION OF A PREFERRED EXAMPLE EMBODIMENT

With reference to the drawings above, 1 indicates as a whole a cardiovascular device according to a first embodiment of the invention, which comprises a flexible frame 2, preferably made of Nitinol®, and a membrane 3 which rests on the frame 2 and is attached thereto, in the manner that will be shown later.

The frame 2 is formed by a plurality of thread-like elements 4, each of which has a first portion 4A which has a first end which, like other first ends of other base portions of other thread-like elements 4, extends from a central hub 5.

Each thread-like element 4 has a second intermediate portion 4B which is helically shaped which is kept distanced from the others and which culminates in a third portion 4C which has a second end opposite the first end which is attached to an attachment element 6 common to other second ends of the other thread-like elements 4 and substantially coaxial with the central hub 5.

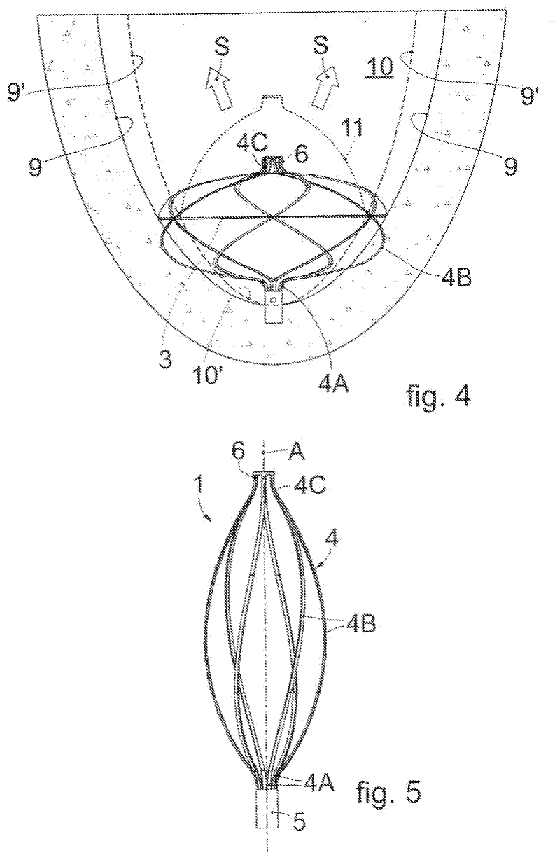

All together the thread-like elements 4 form a plurality of loops 7, substantially equal to each other, which are everted toward the outside and which have the dual function both of creating a surface of the frame 2 with which to support the membrane 3 and allow the constraint thereof to them, and also to rest on the internal walls 9 of a ventricular cavity 10, as shown in FIG. 4, also slightly penetrating in them over time.

As can be seen in particular in FIG. 2, each loop 7 forms, in a view from above, a sort of petal which extends between the hub 5 and the attachment element 6, without however being in contact with the other loops 7, and all together they confer a profile to the frame 2 that has the shape of an ellipsoid or a geoid, that is, of a rotating solid flattened at the poles, with a single central axis of symmetry “A” defined between the hub 5 and the attachment element 6.

With reference again to FIG. 4, it can be noted the behavior of the cardiovascular device 1 in the condition of cardiac systole, indicated with the dotted lines 9′ that show a contraction of the walls 9.

In particular, it can be noted that the profile of the cardiovascular device 1 deforms elastically toward the inside of the ventricular cavity 10, as indicated with the dashed line 11, pushing the volume of blood present therein according to the arrows “S” toward the aortic valve and the aorta (both not visible in the drawings since they are known to the person of skill in the art) and acting as a pump.

The cardiovascular device 1 is implanted inside the left ventricular cavity 10, typically surgically, through an introduction catheter, making it first assume the configuration of minimum transverse bulk with respect to the axis “A” which is visible in FIG. 5 and in which for clarity of representation the membrane 3 has been eliminated.

The introduction and positioning of the vascular device 1 can also occur percutaneously, also using an introduction catheter that reaches the ventricle 5, however through the femoral artery of the patient in which it is inserted until its distal end reaches the ventricular cavity 10.

The cardiovascular device 1 in this step is maintained in the configuration of minimum transverse bulk indicated previously, until it is made to exit from the distal end of the introduction catheter.

After the introduction and the correct positioning, the surgeon fixes the cardiovascular device 1 in position, for example with a suture, and attaches the hub 5 to the bottom of the ventricular cavity 10, indicated with 10′.

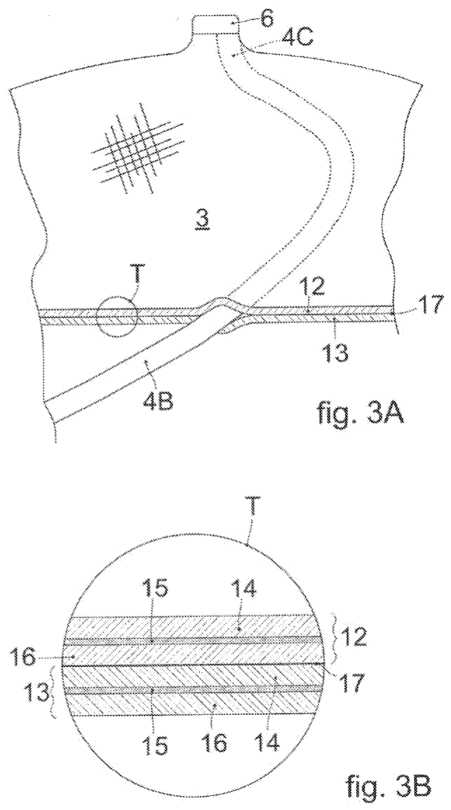

With reference to FIG. 3, it can be noted that the membrane 3 consists of two foils 12 and 13 which are glued together incorporating the portions of the thread-like elements 4 which converge toward the attachment element 6 and, in this way, making the membrane 3 attached to the cardiovascular device 1.

It should be clarified that each foil 12 and 13 that forms the membrane 3 comprises in turn a sandwich assembly which includes a first layer 14 of polytetrafluoroethylene, an intermediate net 15 made of polyethylene terephthalate, a second layer 16 of polytetrafluoroethylene, as indicated in FIG. 3B.

The two foils 12 and 13 are glued together with a layer of adhesive material 17.

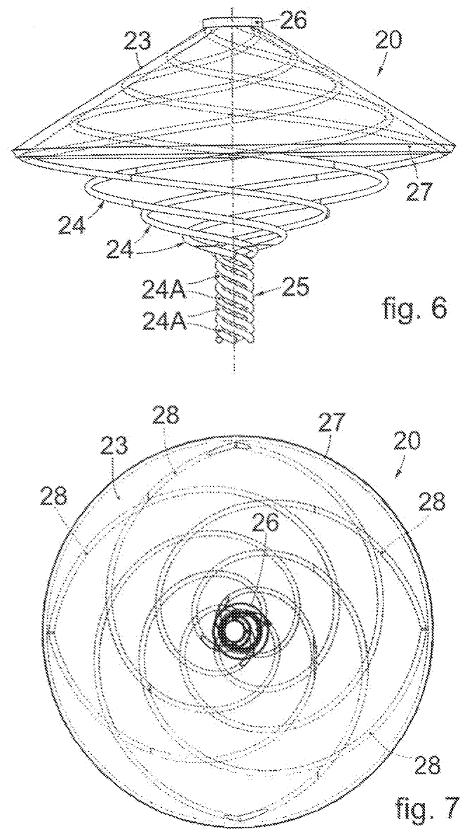

With reference to FIGS. 6 and 7, a second possible embodiment of the cardiovascular device according to the invention is shown, indicated with 20.

As in the version previously described, the cardiovascular device 20 comprises a flexible frame 22 and a membrane 23 which rests on the frame 22 and is attached thereto, in the manner already described previously for the first embodiment.

Also in this second version the frame 22 is preferably made of Nitinol® and is formed by a plurality of flexible thread-like elements 24 which have helical profiles, distanced one from the other and ascending toward a common attachment element 26, and which form, on an opposite part, a hub 25 consisting of a plurality of equal first ends 24A of the thread-like elements 24 which are reciprocally wound together in a helix.

With reference to FIG. 6, the profile which is defined by the whole of all the thread-like elements 24 is a sort of double cone with the vertices opposite and divergent and with a common base that coincides with the external perimeter of the membrane 23 and indicated with 27.

Also in this version, the thread-like elements 24 form a plurality of loops 28 everted toward the outside which define a support and attachment surface of the membrane 23.

This second version also acts like the first version, elastically deforming toward the inside of the ventricular cavity 10 during the systolic phase of the heart and returning in an extended configuration during the diastolic phase.

It should be noted that over time, in both the embodiments 1 and 20 of the cardiovascular device according to the invention, the apical parts of the loops 7 and 28 tend to penetrate into the organic tissue that forms the walls 9 of the ventricular cavity 10, cooperating considerably for the stability of the cardiovascular device itself, which in this way can perform its function both of a diaphragm to reduce the internal volume of the vascular cavity 10, and also of an active pumping element to push the blood in a the cardiocirculatory system of a patient through the aorta, as previously described.

In practice it has been found that the invention achieves the intended purposes.

The invention as conceived is susceptible to modifications and variants, all of which come within the scope of the inventive concept.

Furthermore, all the details can be replaced with other technically equivalent ones.

In practice, any materials, equipment and quantities can be used, according to requirements, without departing from the field of protection of the following claims.

1. A cardiovascular device comprising:
a flexible membrane insertable in a transverse position in a ventricular cavity;
a flexible support and fixing frame of said membrane in said transverse position;
wherein said flexible frame comprises a plurality of shaped thread-like elements each of which comprises a first portion having a first end that is centrally associated with other first ends of other first portions of other thread-like elements thereby forming a central hub, a second portion helically shaped and a third portion having a second end opposite said first end which is attached to an attachment element which is common to other second ends of said other thread-like elements, said thread-like elements forming all together a plurality of loops everted toward the outside to support and constrain said membrane.

2. Device as in claim 1, wherein said loops everted toward the outside shape a three-dimensional ellipsoid/geoid profile.

3. Device as in claim 2, wherein said three-dimensional ellipsoid/geoid profile has a central axis of symmetry.

4. Device as in claim 3, wherein said central axis of symmetry is comprised between said central hub and attachment element.

5. Device as in claim 1, wherein said membrane comprises two superimposed and associated foils of hydrophobic material.

6. Device as in claim 5, wherein each foil of said two superimposed and associated foils comprises a sandwich which includes a first layer of polytetrafluoroethylene, an intermediate net made of polyethylene terephthalate, a second layer of polytetrafluoroethylene.

7. Device as in claim 1, wherein each of said thread-like elements comprises a small arch made of elastically deformable material.

8. Device as in claim 7, wherein said elastically deformable material is Nitinol®.

9. Method to make a flexible support frame of a membrane of a cardiovascular device, comprising:
cutting with a predetermined number of cuts a zone of a bar of flexible material defined between two coaxial monolithic end segments and according to longitudinal and parallel cutting directions, obtaining a plurality of thread-like disjoined elements constrained at their ends;
pressing and rotating said two coaxial monolithic end segments one with respect to the other according to roto-translation portions, thereby obtaining a plurality of said thread-like disjoined elements that shape small flexible arches that define respective everted loops having helicoidal profile; and
blocking said to press and to rotate in a position in which said everted loops form all together a three-dimensional ellipsoid/geoid profile.
